(19) United States
(12) Patent Application Publication
(10) Pub. No.: US 2005/0080112 A1
(43) Pub. Date: Apr. 14, 2005

(54) Compounds for use in disorders associated with mast cell or basophil acitvity

(76) Inventors: Lars Siim Madsen, Smoerum (DK); Bjarne H Dahl, Alleroed (DK)

Correspondence Address: BIRCH STEWART KOLASCH & BIRCH, PO BOX 747, FALLS CHURCH, VA, 22040-0747, US

(21) Appl. No.: 10/481,255

(22) PCT Filed: Jun. 20, 2002
(86) PCT No.: PCT/DK02/00416

(30) Foreign Application Priority Data
Jun. 22, 2001 (DK) PA 2001 00990

(51) Int. Cl. A61K 31/4439; A61K 31/40

(52) U.S. Cl. 514/340; 514/357; 514/422; 514/408

(57) ABSTRACT

The present invention relates to the use certain compounds for the treatment, prevention or alleviation of a disorder or disease of a subject, which disorder or disease is responsive to modulation of the mast cell or basophil activity of such a subject.

The present invention relates to the use certain compounds for the treatment, prevention or alleviation of a disorder or disease of a subject, which disorder or disease is responsive to modulation of the mast cell or basophil activity of such a subject.

### BACKGROUND ART

Mast cells and basophils play a key role in the pathogenesis of several immunological and inflammatory diseases, not only by producing inflammatory and fibrogenic mediators, but also by directly and indirectly secreting various cytokines and chemokines.

Due to the frequency and severity of many of these diseases, there is a continued strong interest in the development of a more selective and effective therapy with fewer side effects for the treatment of the diseases.

A number of chloride channel blockers are described in the international patent applications WO 97/45400, WO 98/47879, WO 00/20378, and WO 00/24707 (all NeuroSearch A/S).

### SUMMARY OF THE INVENTION

In its first aspect, the invention relates to the use of a compound of the general formula I
A-(X)ₚ—(Y)_{q}-(Z)ᵣ-B (I)

or a pharmaceutically acceptable salt or a prodrug thereof for the manufacture of a medicament for the treatment, prevention or alleviation of a disorder or disease of a subject, which disorder or disease is responsive to modulation of the mast cell or basophil activity of such a subject. Other objects of the invention will be apparent to the person skilled in the art from the following detailed description and examples.

### DETAILED DISCLOSURE OF THE INVENTION

We have now found that these compounds can be used for treating a disorder or disease of a living animal body, which is responsive to modulation of the mast cell or basophil activity of such a living animal body.

Thus, in its first aspect, the invention provides the use of a compound of the general formula I
A-(X)ₚ—(Y)_{q}-(Z)ᵣ-B (I)

wherein

A represents a first ring structure selected from aryl, or heteroaryl;

which first ring structure is optionally substituted with one or more substituents independently selected from the group consisting of:

halogen, hydroxy, amino, oxy, cyano, nitro, trifluoromethyl, trifluoromethoxy, trifluorothiomethoxy

alkyl, cycloalkyl, cycloalkylalkyl, alkenyl, alkynyl, alkoxy,

aryl, arylalkyl, aryloxy, arylcarboxy, heteroaryl, —N(R²)-aryl,

a 5- or 6-membered monocyclic heterocyclic group,

—CO₂R¹, —COR¹, -alkyl-CO₂R¹, -alkyl-COR¹,

—N(R²)₂, -alkyl-N(R²)₂, —CO₂N(R¹)₂, —NHCOR¹, —CON(R¹)₂, —NHSO₂R¹, —CONHSO₂R¹, —SO₂N(R¹)₂, and —SO₂OR¹;

wherein each of the alkyl, alkoxy, and cycloalkyl is optionally substituted with one or more substitutents independently selected from the group consisting of:

halogen, hydroxy, amino, cyano, nitro, trifluoromethyl, trifluoromethoxy, trifluorothiomethoxy alkyl, cycloalkyl, cycloalkylalkyl, alkenyl, and alkynyl;

each of the aryl, heteroaryl, and 5- or 6-membered monocyclic heterocyclic group is optionally substituted with one or more one or more substitutents independently selected from the group consisting of:

halogen, hydroxy, amino, cyano, nitro, trifluoromethyl, trifluoromethoxy, trifluorothiomethoxy alkyl, cycloalkyl, cycloalkylalkyl, alkenyl, alkynyl, alkoxy,

aryl, heteroaryl, —CO₂R³, —COR³,

—N(R⁴)₂, -alkyl-N(R⁴)₂, —CON(R³)₂, —NHCOR³, —CON(R³)₂, —NHSO₂R³, —SO₂N(R³)₂, and —SO₂OR³;

each of R¹ and R³ independently is selected from the group consisting of:

hydrogen, alkyl, cycloalkyl, cycloalkylalkyl, alkenyl, alkynyl, aryl, arylalkyl, heteroaryl, and

a 5-8 membered ring optionally containing double bonds and optionally containing one or two heteroatoms, which heteroatoms can be substituted with alkyl or acyl;

or (R¹)₂ or (R³)₂ independently together with the heteroatom to which it is connected represents a 5-8 membered ring optionally containing double bonds and optionally containing another heteroatom, which heteroatom can be substituted with alkyl or acyl;

each of R² and R⁴ independently is hydrogen or alkyl;

B represents a second ring structure

selected from aryl, or heteroaryl;

which second ring structure is substituted with one or more acidic functional group having a pKa value below 8, or a group which is convertible in vivo to such a group, or a bioisostere thereof;

and which second ring structure is furthermore optionally substituted with one or more substituents independently selected from the group consisting of:

halogen, hydroxy, amino, oxy, cyano, nitro, trifluoromethyl, trifluoromethoxy, trifluorothiomethoxy

alkyl, cycloalkyl, cycloalkylalkyl, alkenyl, alkynyl, alkoxy,

aryl, arylalkyl, aryloxy, arylcarboxy, heteroaryl, —N(R⁶)-aryl,

a 5- or 6-membered monocyclic heterocyclic group,

—CO₂R⁵, —COR⁵, -alkyl-CO₂R⁵, -alkyl-COR⁵,

—N(R⁶)₂, -alkyl-N(R⁶)₂, —CON(R⁵)₂, —NHCOR⁵, —CON(R⁵)₂, —NHSO₂R⁵, —CONHSO₂R⁵, —SO₂N(R⁵)₂, and —SO₂OR⁵;

wherein each of the alkyl, alkoxy, and cycloalkyl is optionally substituted with one or more substitutents independently selected from the group consisting of:

halogen, hydroxy, amino, cyano, nitro, trifluoromethyl, trifluoromethoxy, trifluorothiomethoxy alkyl, cycloalkyl, cycloalkylalkyl, alkenyl, and alkynyl;

each of the aryl, heteroaryl, and 5- or 6-membered monocyclic heterocyclic group is optionally substituted with one or more one or more substitutents independently selected from the group consisting of:

halogen, hydroxy, amino, cyano, nitro, trifluoromethyl, trifluoromethoxy, trifluorothiomethoxy alkyl, cycloalkyl, cycloalkylalkyl, alkenyl, alkynyl, alkoxy,

aryl, heteroaryl, —CO₂R⁷, —COR⁷,

—N(R⁸)₂, -alkyl-N(R⁸)₂, —CO₂N(R⁷)₂, —NHCOR⁷, —CON(R⁷)₂, —NHSO₂R⁷, —SO₂N(R⁷)₂, and —SO₂OR⁷;

each of R⁵ and R⁷ independently is selected from the group consisting of:

hydrogen, alkyl, cycloalkyl, cycloalkylalkyl, alkenyl, alkynyl, aryl, arylalkyl, heteroaryl, and

a 5-8 membered ring optionally containing double bonds and optionally containing one or two heteroatoms, which heteroatoms can be substituted by alkyl or acyl;

or (R⁵)₂ or (R⁷)₂ independently together with the heteroatom to which it is connected represents a 5-8 membered ring optionally containing double bonds and optionally containing another heteroatom, which heteroatom can be substituted by alkyl or acyl;

each of R⁶ and R⁸ independently is hydrogen or alkyl;

X, Y, and Z are independently selected from the group consisting of:

—CO—, —CS—, —SO₂—, —C(═NR⁹)—, —NR¹⁰—, —(CH₂)ₛ—, —O—,

—CH₂—NH—, —SO₂—NH—, —CH═CH—, —C≡C—, and —N═CH—;

wherein s is 1, 2, or 3;

R⁹ is hydrogen, alkyl, or cyano;

R¹⁰ is hydrogen or alkyl;

p, q, and r independently are 0 or 1;

the sum p+q+r is 1, 2, or 3;

or —(X)ₚ—(Y)_{q}-(Z)ᵣ- represents

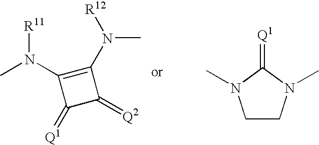

wherein

Q¹ and Q² independently represent O or S;

R¹¹ and R¹² independently are hydrogen or alkyl;

or a pharmaceutically acceptable salt or a prodrug thereof for the manufacture of a medicament for the treatment, prevention or alleviation of a disorder or disease of a subject, which disorder or disease is responsive to modulation of the mast cell or basophil activity of such a subject.

In a further aspect, the invention provides a method of treatment, prevention or alleviation of a disorder or disease of a subject, which disorder or disease is responsive to modulation of the mast cell or basophil activity of such a subject, which method comprises administering to said subject a therapeutically effective amount of a compound of general formula I or a pharmaceutically acceptable salt or a prodrug thereof.

In one embodiment, the acidic functional group having a pKa below 8, or a group which is convertible in vivo to such a group is selected from the group consisting of:

—COOH, —CH₂CO₂R¹³, —CON(R¹³)₂, tetrazolyl, methyltetrazolyl, 3-oxo-1,2-dihydro-1,2,4-triazolyl, 2-oxo-3H-1,3,4-oxadiazolyl, 3-oxo-1,2-dihydro-1,2,4-triazolyl, 4-hydro-1,2,4-triazolyl, —NHSO₂R¹³, —CO₂R¹³, —CO₂N(R¹³)₂, —SO₂OR¹³, —SO₂N(R¹³)₂, —CONHOH, —CONHNH₂, —CONHSO₂R¹³, —CONHSO₂OR³, —PO(OR¹³)₂, and —SO₂)R¹³;

wherein each of R¹³ independently is selected from the group consisting of:

hydrogen, alkyl, cycloalkyl, cycloalkylalkyl, alkenyl, alkynyl, aryl, aralkyl, and heteroaryl;

or R¹³ comprises a 5-8 membered ring optionally containing double bonds and optionally containing one or two heteroatoms, which heteroatoms can be substituted by alkyl or acyl;

or (R¹³)₂ together with the heteroatom to which it is connected represents a 5-8 membered ring optionally containing double bonds and optionally containing another heteroatom, which heteroatoms can be substituted by alkyl or acyl.

In a second embodiment, the bioisostere of the acidic functional group is two neighbouring fluoro.

In a third embodiment, the second ring structure is substituted with an acidic functional group having a pKa below 8, or a group which is convertible in vivo to such a group, or a bioisostere thereof, in the position nearest or second nearest to the position attached to —(X)ₚ—(Y)_{q}-(Z)ᵣ-.

In a further embodiment, the acidic functional group having a pKa below 8, or a group which is convertible in vivo to such a group is selected from the group consisting of:

—COOH, —CH₂CO₂R¹³, —CON(R¹³)₂, tetrazolyl, methyltetrazolyl, —NHSO₂R¹³, —CO₂R¹³, —CO₂N(R¹³)₂, —SO₂N(R¹³)₂, —CONHSO₂R¹³, —PO(OR¹³)₂, and —SO₂OR¹³;

wherein R¹³ is as defined above.

In a still further embodiment, the first ring structure is optionally substituted with one or more substituents independently selected from the group consisting of:

trifluoromethyl, halogen, alkyl, alkoxy, nitro, —COR¹, —COOH, —CH₂CO₂R¹, —CON(R¹)₂, —NHSO₂R¹, —NHCOR¹, —CO₂R¹, —CO₂N(R¹)₂, —SO₂N(R¹)₂, —CONHSO₂R¹, —SO₂OR¹, and aryl;

wherein the aryl optionally is substituted with one or more substituents selected from the group:

—NO₂, —NHCOR³, —CO₂R³, —CON(R³)₂, —NHSO₂R³, and —SO₂N(R³)₂;

wherein R¹ and R³ are defined as above.

In a further embodiment, the second ring structure is substituted with one or more acidic functional group having a pKa value below 8, or a group which is convertible in vivo to such a group, or a bioisostere thereof;

and which second ring structure is furthermore optionally substituted with one or more substituents independently selected from the group consisting of:

alkyl, nitro, amino, alkylamino, CO₂R⁹, CF₃, alkyl, halogen, hydroxy, alkoxy, —NHCOR⁵, —N(R⁵)₂, —CON(R⁵)₂, and aryl,

wherein the aryl is optionally substituted with one or more substituents independently selected from the group consisting of:

—NO₂, —CON(R⁷)₂, —NHCOR⁷, —SO₂N(R⁷)₂, and —CO₂R⁷;

wherein R⁵ and R⁷ are defined as above.

In a still further embodiment, X is —NR⁹—, Y is —CO— or —CS—, Z is —NR¹⁰—, p is 1, q is 1 and r is 1; wherein R¹⁰ is defined as above.

In a further embodiment, Y is —CO— or —CS—, Z is —NR¹⁰—, p is 0, q is 1, and r is 1.

In a still further embodiment, X is —CH₂—, Y is —CH₂—, Z is —NR¹⁰—, p is 1, q is 1 and r is 1.

In a further embodiment, X is —NR¹⁰—, Y is —SO₂—, Z is —NR¹⁰—, p is 1, q is 1 and r is 1.

In a still further embodiment, X is —CH₂—NH—, Y is —CO— or —CS—, Z is —NR¹⁰—, p is 1, q is 1 and r is 1.

In a further embodiment, X is —O—, Y is —CO—, Z is —NR¹⁰—, p is 1, q is 1 and r is 1.

In a still further embodiment, X is —SO₂—NH—, Y is —CO—, Z is —NH—, p is 1, q is 1 and r is 1.

In a further embodiment, X is —NR¹⁰—, Y is —(CH₂)ₛ—, Z is —NR¹⁰—, p is 1, q is 1 and r is 1; wherein s is defined as above.

In a special embodiment, R¹⁰ is hydrogen.

In a further embodiment, s is 2.

In a still further embodiment, —(X)ₚ—(Y)_{q}-(Z)ᵣ- represents

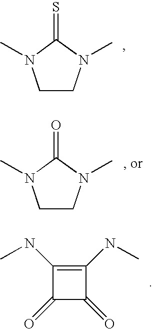

In a further embodiment, the first ring structure is phenyl, naphthyl, indanyl, or pyridyl.

In a still further embodiment, the second ring structure is phenyl, naphthyl, indanyl or pyridyl.

In a special embodiment, the first ring structure is phenyl, the second ring structure is phenyl, and —(X)ₚ—(Y)_{q}-(Z)ᵣ- represents —NH—CO—NH—.

In a further special embodiment, the first ring structure is phenyl, the second ring structure is phenyl, —(X)ₚ—(Y)_{q}-(Z)ᵣ- represents —NH—CO—NH—, and the acidic functional group having a pKa below 8 is —COOH or tetrazolyl, and the bioisostere of the acidic functional group having a pKa below 8 is two neighbouring fluoro.

In a still further special embodiment, A represents 3-biphenylyl, 3-chlorophenyl, 3-nitrophenyl, 3-trifluoromethylphenyl, 3,5-bis(trifluoromethyl)phenyl, 3,4-dichlorophenyl, 4-chloro-3-trifluoromethylphenyl, or 4-fluoro-3-trifluoromethylphenyl.

In a further special embodiment, B represents 2-(1-H-tetrazol-5-yl)phenyl, 2-carboxy-5-chlorophenyl, 2-carboxy-5-fluorophenyl, 2-carboxy-5-nitrophenyl, 5-chloro-2-(1-H-tetrazol-5-yl )phenyl, 2,3,4-trifluorophenyl, 2,3-difluorophenyl, 2,4-dibromo-6-(1-H-tetrazol-5-yl)phenyl, or 4-bromo-2-(1-H-tetrazol-5-yl)phenyl.

In a special embodiment, the compound of general formula I is selected from:

3-Trifluoromethylphenyl-N′-2-carboxyphenyl urea

N-3-Trifluoromethylphenyl-N′-3-carboxyphenyl urea;

N-(2-Methoxy-5-chlorophenyl)-N′-3-carboxyphenyl urea;

N-3-Trifluoromethylphenyl-N′-(2-carboxy-5-nitrophenyl) urea;

N-3-Trifluoromethylphenyl-N′-(2-carboxy-4-methylphenyl) urea;

N-3-Trifluoromethylphenyl-N′-(4-bromo-2-carboxyphenyl) urea;

N-3-Trifluoromethylphenyl-N′-3-carbamoylphenyl urea;

N-3-Trifluoromethylphenyl-N′-3-sulfamoylphenyl urea;

N-3-Trifluoromethylphenyl-N′-(5-chloro-2-phenylsulfonamidocarbonylphenyl) urea;

N-3-Trifluoromethylphenyl-N′-2-methylsulfonamidocarbonylphenyl urea;

N-3-Trifluoromethylphenyl-N′-(6-methyl-2-carboxyphenyl) urea;

N-3-Trifluoromethylphenyl-N′-(3-methyl-2-carboxyphenyl) urea;

N-3-Trifluoromethylphenyl-N′-(4-hydroxy-2-carboxyphenyl) urea;

N-4-Nitrophenyl-N′-2-carboxyphenyl urea;

N-3-Trifluoromethylphenyl-N′-2-carboxymethylphenyl urea;

N-3-Trifluoromethylphenyl-N′-2-sulfophenyl urea;

N-3-Trifluoromethylphenyl-N′-2-carboxyphenyl thiourea;

N-3-Trifluoromethylphenyl-N′-(2-carboxy-5-trifluoromethylphenyl) urea;

N-3-Trifluoromethylphenyl-N′-(4,5-dimethoxy-2-carboxyphenyl) urea;

N-3-Carboxyphenyl-N′-(2-hydroxy-5-chlorophenyl) urea;

N-3-Carbamoylphenyl-N′-(2-hydroxy-5-chlorophenyl) urea;

N-3-Trifluoromethylphenyl-N′-(2-hydroxy-4-nitro-5-carboxyphenyl) urea;

N-3-Trifluoromethylphenyl-N′-(4-carboxy-5-chloro-2-hydroxyphenyl) urea;

N-3-Trifluoromethylphenyl-N′-(2-amino-5-chlorophenyl) urea;

N-3-Trifluoromethylphenyl-N′-(5-chloro-2-methanesulfonylaminophenyl) urea;

N-3-Trifluoromethylphenyl-N′-2-carboxyphenyl urea isopropyl ester;

N-3-Trifluoromethylphenyl-N′-2-carboxyphenyl urea methyl ester;

N-3-Trifluoromethylphenyl-N′-2-hydrazinocarbonylphenyl urea;

N-3-Trifluoromethylphenyl-N′-2-hydroxylaminocarbonylphenyl urea;

2-(3′-Trifluoromethylbenzylcarboxamido)benzoic acid;

N-3-Trifluoromethylphenyl-N′-4-carboxyphenyl urea;

N-3-Trifluoromethylphenyl-N′-(2-carboxy-4-nitrophenyl) urea;

N-3-Trifluoromethylphenyl-N′-2-carboxynapht-3-yl urea;

N-3-Trifluoromethylphenyl-N′-(4-methoxy-2-carboxyphenyl) urea;

N-3-Methoxyphenyl-N′-2-carboxyphenyl urea;

N-4-Bromophenyl-N′-2-carboxyphenyl urea;

N-3-Nitrophenyl-N′-2-carboxyphenyl urea;

N-2-Methoxyphenyl-N′-2-carboxyphenyl urea;

N-4-Methoxyphenyl-N′-2-carboxyphenyl urea;

N-1-Naphthyl-N′-2-carboxyphenyl urea;

N-2-Trifluoromethyl phenyl-N′-2-carboxyphenyl urea;

N-4-Methylphenylsulfonyl-N′-2-carboxyphenyl urea;

N-3-Trifluoromethylphenyl-N′-(2-ethyloxycarbonylphenyl)-1,2-diaminoethane;

N-(3-Trifluoromethyl)phenyl-N′-(2-carboxy)phenylsulfamide;

N-3-Trifluoromethylbenzyl-N′-2-carboxyphenyl urea;

N-(3-Trifluoromethyl-4-phenylphenyl)-N′-2-carboxyphenyl urea;

2-(3′-Trifluoromethylphenyloxycarbonylamino)benzoic acid;

N-3-Trifluoromethylphenyl-N′-(5-chloro-2-aminophenyl) urea;

N-3-Trifluoromethylphenyl-N′-[4-nitro-2-(1-H-tetrazol-5-yl)phenyl] urea;

N-3-Trifluoromethylphenyl-N′-[4-(2-naphthyl)-2-(1-H-tetrazol-5-yl)phenyl] urea;

N-3-Trifluoromethylphenyl-N′-[4-(3-pyridyl)-2-(1-H-tetrazol-5-yl)phenyl] urea;

N-3-Trifluoromethylphenyl-N′-[4-(1-naphthyl)-2-(1-H-tetrazol-5-yl)phenyl] urea;

N-3-Trifluoromethylphenyl-N′-[4-(4-trifluoromethylphenyl)-2-(1-H-tetrazol-5-yl)phenyl] urea;

N-3-Trifluoromethylphenyl-N′-[4-(3-furyl)-2-(1-H-tetrazol-5-yl)phenyl] urea;

N-3-Trifluoromethylphenyl-N′-[4-(3-thienyl)-2-(1-H-tetrazol-5-yl)phenyl] urea;

N-3-Trifluoromethylphenyl-N′-[4-(3-nitrophenyl)-2-(1-H-tetrazol-5-yl)phenyl] urea;

N-3-Trifluoromethylphenyl-N′-[4-(4-ethoxycarbonylphenyl)-2-(1-H-tetrazol-5-yl)phenyl] urea;

N-3-Trifluoromethylphenyl-N′-[4-(4-dimethylaminocarbonylphenyl)-2-(1-H-tetrazol-5-yl)phenyl] urea;

N-3-Trifluoromethylphenyl-N′-[4-(4-aminocarbonylphenyl)-2-(1-H-tetrazol-5-yl)phenyl] urea;

N-3-Trifluoromethylphenyl-N′-2-(4-hydroxy-1,2,4-triazol-3-yl)phenyl urea;

N-3-Trifluoromethylphenyl-N′-2-(3-oxo-1,2-dihydro-1,2,4-triazol-1-yl)phenyl urea;

N-3-Trifluoromethylphenyl-N′-2-(2-oxo-3H-1,3,4-oxadiazol-5-yl)phenyl urea;

N-3-Trifluoromethylphenyl-N′-[5-phenyl-2-(3-oxo-1,2-dihydro-1,2,4-triazol-1-yl)phenyl] urea;

N-3-Trifluoromethylphenyl-N′-[4-amino-2-(1-H-tetrazol-5-yl)phenyl] urea;

N-3-Trifluoromethylphenyl-N′-[4-acetylamino-2-(1-H-tetrazol-5-yl)phenyl] urea;

N-3-Trifluoromethylphenyl-N′-[4-benzoylamino-2-(1-H-tetrazol-5-yl)phenyl] urea;

N-3-Trifluoromethylphenyl-N′-[4-(4-carboxyphenyl)-2-(1-H-tetrazol-5-yl)phenyl] urea;

N-3-Trifluoromethylphenyl-N′-[4-(4-aminocarbonylphenyl)-2-(1-H-tetrazol-5-yl)phenyl] urea;

N-4-Biphenylyl-N′-2-(1-H-tetrazol-5-yl)phenyl urea;

N-3-Biphenylyl-N′-2-(1-H-tetrazol-5-yl)phenyl urea;

N-5-Indanyl-N′-2-(1-H-tetrazol-5-yl)phenyl urea;

N-3-Bromophenyl-N′-[4-bromo-2-(1-H-tetrazol-5-yl)phenyl] urea;

N-3-Acetylphenyl-N′-2-(1-H-tetrazol-5-yl)phenyl urea;

N-3-Biphenylyl-N′-[4-bromo-2-(1-H-tetrazol-5-yl)phenyl] urea;

N-3-(3-Pyridyl)phenyl-N′-[4-bromo-2-(1-H-tetrazol-5-yl)phenyl] urea;

N-3-Trifluoromethylphenyl-N′-[4-bromo-2-(1-H-tetrazol-5-yl)phenyl urea];

N-3-Trifluoromethylphenyl-N′-2-(1-H-tetrazol-5-yl)phenyl urea;

N-3-Trifluoromethylphenyl-N′-2-(1-H-tetrazol-5-yl)phenyl thiourea;

N-3-Trifluoromethylphenyl-N′-[4-phenyl-2-(1-H-tetrazol-5-yl)phenyl] urea;

N-4-Trifluoromethylphenyl-N′-2-(1-H-tetrazol-5-yl)phenyl urea;

N-3-Chlorophenyl-N′-2-(1-H-tetrazol-5-yl)phenyl urea;

N-Phenyl-N′-2-(1-H-tetrazol-5-yl)phenyl urea;

N-3-Bromophenyl-N′-[4-bromo-2-(1-H-tetrazol-5-yl)phenyl] urea;

3-[4-Bromo-2-(1-H-tetrazol-5-yl)-phenylamino]-4-(3-trifluoromethyl-phenylamino)-3-cyclobuten-1,2-dione;

3-(3-Bromo-phenylamino)-4-[4-bromo-(1-H-tetrazol-5-yl)-phenylamino]-3-cyclobuten-1,2-dione;

3-(3-Bromo-phenylamino)-4-[4′-(N,N-dimethyl sulfonamide)-2-(1-H-tetrazol-5-yl)-biphenylamino]-3-cyclobuten-1,2-dione;

3-(3-Bromo-phenylamino)-4-[2-(1-H-tetrazol-5-yl)-biphenylamino]-3-cyclobuten-1,2-dione;

N-Phenyl-N′-(2-carboxyphenyl) urea;

N-3-Trifluoromethylphenyl-N′-(2-carboxyphenyl)-N′-methyl urea;

N-3-Trifluoromethylphenyl-N′-(4-bromo-2-carboxyphenyl) urea;

N-3-Trifluoromethylphenyl-N′-(2-carboxy-4-chlorophenyl) urea;

N-3-Trifluoromethylphenyl-N′-(2-carboxy-4-fluorophenyl) urea;

N-3-Trifluoromethylphenyl-N′-(5-bromo-2-carboxyphenyl) urea;

N-3-Trifluoromethylphenyl-N′-(2-carboxy-5-chlorophenyl) urea;

N-3-Bromophenyl-N′-[2-(1-H-tetrazol-5-yl)-4-biphenyl] urea;

N-3-Trifluoromethylphenyl-N′-[4′-(N,N-dimethylsulfamoyl)-2-(1-H-tetrazol-5-yl)-4-biphenyl] urea;

N-3-Bromophenyl-N′-[4′-(N,N-dimethylsulfamoyl)-2-(1-H-tetrazol-5-yl)-4-biphenyl] urea;

N-3-Bromophenyl-N′-[4′-(N,N-dimethylcarbamoyl)-2-(1-H-tetrazol-5-yl)-4-biphenyl] urea;

N-3-Trifluoromethylphenyl-N′-[4-amino-2-(1-H-tetrazol-5-yl)phenyl] urea;

N-3-Trifluoromethylphenyl-N′-[4-acetylamino-2-(1-H-tetrazol-5-yl)phenyl] urea;

N-3-Trifluoromethylphenyl-N′-[4 ′-carbamoyl-2-(1-H-tetrazol-5-yl)-4-biphenyl] urea;

N-3-Trifluoromethylphenyl-N′-[4′-(N,N-dimethylcarbamoyl)-2-(1-H-tetrazol-5-yl)-4-biphenyl] urea;

N-3-Trifluoromethylphenyl-N′-[4′-carboxy-2-(1-H-tetrazol-5-yl)-4-biphenyl] urea;

1-(3-Trifluoromethylphenyl)-3-(2-carboxyphenyl)-2-imidazolidone;

N-3-Trifluoromethylphenyl-N′-[4-(4-benzoylcarbonyl phenyl)-2-(1-H-tetrazol-5-yl)phenyl] urea;

N-4-Biphenylyl-N′-2-(1-H-tetrazol-5-yl)phenyl urea;

N-3-Bromophenyl-N′-[3′-nitro-2-(1-H-tetrazol-5-yl)biphenyl] urea;

N-3-Bromophenyl-N′-[4′-(sulfoamido-N-methylpiperazinium chloride)-2-(1-H-tetrazol-5-yl)-4-biphenyl] urea;

N-3-Bromophenyl-N′-[4′-(carbamoyl-N-methylpiperazine)-2-(1-H-tetrazol-5-yl)-4-biphenyl] urea;

N-3,5-Dichlorophenyl-N′-[4-bromo-2-(1-H-tetrazol-5-yl)phenyl] urea;

N-4-Trifluoromethylphenyl-N′-[4-bromo-2-(1-H-tetrazol-5-yl)phenyl] urea;

N-4-Bromophenyl-N -[4-bromo-2-(1-H-tetrazol-5-yl)phenyl] urea;

N-3-Methoxyphenyl-N′-[4-bromo-2-(1-H-tetrazol-5-yl)phenyl] urea;

N-3-Chlorophenyl)-N′-[4-bromo-2-(1-H-tetrazol-5-yl)phenyl] urea;

N-3-Methylphenyl-N′-[4-bromo-2-(1-H-tetrazol-5-yl)phenyl] urea;

N-3,4-Dichlorophenyl-N′-[4-bromo-2-(1-H-tetrazol-5-yl)phenyl] urea;

N-2-Naphthyl-N′-[4-bromo-2-(1-H-tetrazol-5-yl)phenyl] urea;

N-(4-Methyl-3-nitrophenyl)-N′-[4-bromo-2-(1-H-tetrazol-5-yl)phenyl] urea;

N-(2-Chloro-4-trifluoromethylphenyl)-N′-[4-bromo-2-(1-H-tetrazol-5-yl)phenyl] urea;

N-3,5-Di(trifluoromethyl)phenyl-N′-[4-bromo-2-(1-H-tetrazol-5-yl)phenyl] urea;

N-3,5-Dimethylphenyl-N-[4-bromo-2-(1-H-tetrazol-5-yl)phenyl] urea;

N-4-Ethoxyphenyl-N′-[4-bromo-2-(1-H-tetrazol-5-yl)phenyl] urea;

N-4-Methoxyphenyl-N′-[4-bromo-2-(1-H-tetrazol-5-yl)phenyl] urea;

N-2-Trifluoromethylphenyl-N′-[4-bromo-2-(1-H-tetrazol-5-yl)phenyl] urea;

N-2-Bromophenyl-N′-[4-bromo-2-(1-H-tetrazol-5-yl)phenyl] urea;

N-2-Chlorophenyl-N′-[4-bromo-2-(1-H-tetrazol-5-yl)phenyl] urea;

N-2-Fluorophenyl-N′-[4-bromo-2-(1-H-tetrazol-5-yl)phenyl] urea;

N-(4-Chloro-3-trifluoromethylphenyl)-N′-[4-bromo-2-(-H-tetrazol-5-yl)phenyl] urea;

N-3-Bromophenyl-N-2,3-difluorophenyl urea;

N-2-Methylphenyl-N′-[4-bromo-2-(1-H-tetrazol-5-yl)phenyl] urea;

N-2-Ethylphenyl-N′-[4-bromo-2-(1-H-tetrazol-5-yl)phenyl] urea;

N-4-Methylphenyl-N′-[4-bromo-2-(1-H-tetrazol-5-yl)phenyl] urea;

N-2-Nitrophenyl-N′-[4-bromo-2-(1-H-tetrazol-5-yl)phenyl] urea;

N-3-Fluorophenyl-N-[4-bromo-2-(1-H-tetrazol-yl)phenyl] urea;

N-4-(2-Propyl)phenyl-N′-[4-bromo-2-(1-H-tetrazol-5yl)phenyl] urea;

N-3-Nitrophenyl-N′-[4-bromo-2-(1-H-tetrazol-5-yl)phenyl] urea;

N-3-Acetylphenyl-N′-[4-bromo-2-(1-H-tetrazol-5-yl)phenyl] urea;

N-4-Nitrophenyl-N′-[4-bromo-2-(1-H-tetrazol-5-yl)phenyl] urea;

N-3-Trifluoromethylphenyl-N′-2-carboxyphenyl urea;

N-Phenyl-N′-2-carboxyphenyl urea;

N-3-Trifluoromethylphenyl-N′-2-carboxyphenyl-N-methyl urea;

N-3-Trifluoromethylphenyl-N′-[4′-(N-phenylcarbamoyl)-2-(1-H-tetrazol-5-yl)-4-biphenyl] urea;

N-(2-Indan)-N -2-(1-H-tetrazol-5-yl)phenyl urea;

N-(4-Biphenyl)-N′-2-(1-H-tetrazol-5-yl)phenyl urea;

N-(3-Biphenyl)-N′-2-(1-H-tetrazol-5-yl)phenyl urea;

N-(3-Acetylphenyl)-N′-2-(1-H-tetrazol-5-yl)phenyl urea;

N-3-Trifluoromethylphenyl-N′-[2-(1-methyltetrazol-5-yl)-4-biphenyl] urea;

N-(3-Biphenyl)-N-[4-bromo-2-(1-H-tetrazol-5-yl)phenyl] urea;

N-3-(3-Pyridyl)phenyl-N′-[4-bromo-2-(1-H-tetrazol-5-yl)phenyl] urea hydrochloride;

N-3-Bromophenyl-N′-[4-bromo-2-(1-H-tetrazol-5-yl)phenyl] urea;

1-(3-Trifluoromethylphenyl)-3-(2-carboxyphenyl)-2-imidazolidone;

N-(4-Biphenylyl)-N′-2-(1-H-tetrazol-5-yl)phenyl urea;

N-(3-Biphenylyl)-N′-2-(1-H-tetrazol-5-yl)phenyl urea;

N-(5-Indanyl)-N′-2-(1-H-tetrazol-5-yl)phenyl urea;

N-(2-Chloro-5-trifluoromethylphenyl)-N′-[4-bromo-2-(1-H-tetrazol-5-yl)phenyl] urea;

N-4-Bromophenyl-N′-(2-carboxy-5-chlorophenyl) urea;

N-4-Trifluoromethylphenyl-N′-(2-carboxy-5-chlorophenyl) urea;

N-3-Bromophenyl-N′-(2-carboxy-5-chlorophenyl) urea;

N-3-Nitrophenyl-N′-(2-carboxy-5-chlorophenyl) urea;

N-3-Methoxyphenyl-N′-(2-carboxy-5-chlorophenyl) urea;

N-(4-Chloro-3-trifluoromethylphenyl)-N′-(2-carboxy-5-chlorophenyl) urea;

N-3-Fluorophenyl-N′-(2-carboxy-5-chlorophenyl) urea;

N-3-Fluorophenyl-N′-(2-carboxy-5-fluorophenyl) urea;

N-3-Trifluoromethylphenyl-N′-(2-carboxy-4,5-difluorophenyl) urea;

N-3,5-Bis(trifluoromethyl)phenyl)-N′-(2-carboxy-5-chlorophenyl) urea;

N-3-Trifluoromethylphenyl-N′-(2-carboxy-5-nitrophenyl) urea;

N-3,4-Dichlorophenyl-N′-[5-methyl-2-(1-H-tetrazol-5-yl)phenyl] urea;

N-3,4-Dichlorophenyl-N′-[5-chloro-2-(1-H-tetrazol-5-yl)phenyl] urea;

N-3-Trifluoromethylphenyl-N′-(3-carboxy-4-chlorophenyl) urea;

N-(3-Chloro-4-hydroxyphenyl)-N′-[4-bromo-2-(1-H-tetrazol-5-yl)phenyl] urea;

N-2,3,4-Trifluorophenyl-N′-[4-bromo-2-(1-H-tetrazol-5-yl)phenyl] urea;

N-3,4-Difluorophenyl-N′-[4-bromo-2-(1-H-tetrazol-5yl)phenyl] urea;

N-3-Chlorophenyl-N′-2,3-difluorophenyl urea;

N-(3-Chloro-4-fluorophenyl)-N′-[4-bromo-2-(1-H-tetrazol-5-yl)phenyl] urea;

N-2,4,5-Trifluorophenyl-N′-[4-bromo-2-(1-H-tetrazol-5-yl)phenyl] urea;

N-3,5-Bis(trifluoromethyl)phenyl)-N′-2-(1-H-tetrazol-5-yl)phenyl urea;

N-3,5-Bis(trifluoromethyl)phenyl)-N′-[2,4-dibromo-6-(1-H-tetrazol-5-yl)phenyl] urea;

N-(4-Fluoro-3-trifluoromethylphenyl)-N′-[4-bromo-2-(1-H-tetrazol-5-yl)phenyl] urea;

N-3,5-Difluorophenyl-N′-[4-bromo-2-(1-H-tetrazol-5-yl)phenyl] urea;

N-3,5-Bis(trifluoromethyl)phenyl-N′-[4′-(N,N-dimethylsulfamoyl)-2-(1-H-tetrazol-5-yl)-(4-biphenyl)] urea;

N-3,5-Dichlorophenyl-N′-[4′-(N,N-dimethylsulfamoyl)-2-(1-H-tetrazol-5-yl)-4-biphenyl] urea;

N-2,3-Difluorophenyl-N′-3-trifluoromethylphenyl urea;

N-2, 3-Difluorophenyl-N′-(4-chloro-3-trifluoromethylphenyl) urea;

N-3,4-Dichlorophenyl-N′-2,3-trifluorophenyl urea;

N-2,3-Difluorophenyl-N′-3-trifluoromethylphenyl thiourea;

N-2,3-Difluorophenyl-N′-2-fluorophenyl urea;

N-2,3-Difluorophenyl-N′-3-methoxyphenyl urea;

N-3,4-Dichlorophenyl-N′-2,3,4-trifluorophenyl urea;

N-(4-Chloro-3-trifluoromethylphenyl)-N′-2,3,4-trifluorophenyl urea;

N-3-Chlorophenyl-N′-(2-hydroxy-4-methylphenyl) urea;

N-2,3-Difluorophenyl-N′-[3′-(pyridin-3-yl)phenyl] urea;

N-3,5-Dichlorophenyl-N′-2,3-difluorophenyl urea;

N-2,3-Difluorophenyl-N′-3-nitrophenyl urea; and

pharmaceutically acceptable salts and prodrugs thereof.

The above compounds and their preparation are described in WO97/45400, WO98/47879, WO00/20378, and WO00/24707.

In one embodiment, the compound of general formula I show an inhibition of more than 10%, preferably more than 25%, and most preferably more than 50%, when tested for In vitro inhibition of anti-IgE induced basophil histamine release (example 1).

The disorders or diseases to be treated include, but are not limited to, disorders or diseases responsive to modulation of mast cell or basophil production or secretion of mediators such as histamine, neutral proteases or tryptases (such as chymotryptases and carboxypeptidases), leukotrienes (such as LTC4, and LTB4), prostaglandins (such as PGD2), TXA2, PAF, and cytokines (such as IL-4 and TNF-α).

In a further embodiment, the disorder or disease that is responsive to modulation of the mast cell or basophil activity is a disorder or disease that is responsive to modulation of mast cell or basophil production or secretion of histamine.

In a still further embodiment, the disorder or disease that is responsive to modulation of the mast cell or basophil activity is allergic bronchopulmonary aspergillosis (ABPA), allergic rhinitis, allergic skin disease, allergic skin reaction, drug induced allergic skin reaction, anaphylaxis, asthma, atherosclerosis, atopic dermatitis (AD), bronchial asthma, cancer, chronic obstructive pulmonary disease (COPD), Chrohn's disease, contact dermatitis, dilated cardiomyopathy, fatal asthma, graft rejection, hypersensitivity pneumonitis, ischemic hearth disease, pulmonary fibrosis, rheumatoid arthritis, systemic sclerosis, urticaria, or uveoretinitis.

In a special embodiment, the disorder or disease that is responsive to modulation of the mast cell or basophil activity is allergic bronchopulmonary aspergillosis (ABPA), allergic rhinitis, allergic skin disease, allergic skin reaction, drug induced allergic skin reaction, asthma, bronchial asthma, fatal asthma or chronic obstructive pulmonary disease (COPD). In a further special embodiment, the disorder or disease is asthma, bronchial asthma, fatal asthma or chronic obstructive pulmonary disease (COPD). In a further special embodiment, the disorder or disease is COPD. In a still further special embodiment, the disorder or disease is asthma.

Definition of Substituents

In the context of this invention halogen represents a fluorine, a chlorine, a bromine or an iodine atom.

Alkyl means a straight chain or branched chain of one to six carbon atoms, including but not limited to, methyl, ethyl, propyl, isopropyl, butyl, isobutyl, t-butyl, pentyl, and hexyl; methyl, ethyl, propyl and isopropyl are preferred groups.

Alkoxy is O-alkyl, wherein alkyl is as defined above.

Acyl is —CO-alkyl wherein alkyl is as defined above.

Aryl is a carbocyclic aromatic ring system such as phenyl, naphthyl (1-naphthyl or 2-naphthyl), indanyl, and indenyl.

The acidic functional group having a pKa below 8 or a group which is converted in vivo to such group are groups such as 3-hydroxy-4-oxo-pyranyl, 2-hydroxy-4-oxo-pyrimidyl, 3,5-dioxo-1,2,4-oxadiazolidinyl, 2,4-dioxo-imidazolidinyl, 2,5-dioxo-3-hydroxy-pyrrolyl, 2,5-dioxo-pyrrolidinyl, 2,4-dioxo-1,3-thiazolidinyl, 3-hydroxy-isoxazolyl, 5-hydroxy-isoxazolyl, 3-hydroxy-isothiazolyl, 3-hydroxy-1,2,5-thiadiazolyl, tetrazolyl, 1-methyltetrazolyl, 3-hydroxy-triazolyl, 3-hydroxy-pyrazolyl, 2-hydroxy-1,3,4-oxadiazolyl, 3-oxo-1,2-dihydro-1,2,4-triazolyl, 2-oxo-3H-1,3,4-oxadiazolyl, 4-hydroxy-1,2,4-triazolyl, 2-hydroxy-1,3,4-oxadiazolyl or 2-hydroxy-3,4-dioxo-cyclobutenyl, NH₂, —N(R¹³)₂, —OR³, —CO₂R¹³, —CH₂CO₂R¹³, —CON(R¹³)₂, —NHSO₂R¹³, —SO₂N(R¹³)₂, —SO₂OR^{1 3}, —SO₂R¹³, —PO(OR¹³)₂, —PO₃H₂, —PO₃R²H, —PO₂NH₂, —CONHOH, —CONHCN, —CONHSO₂R¹³, —CONHSO₂OR¹³ and —CONHNH₂;

wherein each of R¹³ independently is selected from the group consisting of:

hydrogen, alkyl, cycloalkyl, cycloalkylalkyl, alkenyl, alkynyl, aryl, aralkyl, and heteroaryl;

or R¹³ comprises a 5-8 membered ring optionally containing double bonds and optionally containing one or two heteroatoms, which heteroatoms can be substituted by alkyl or acyl;

or (R¹³)₂ together with the heteroatom to which it is connected represents a 5-8 membered ring optionally containing double bonds and optionally containing another heteroatom, which heteroatoms can be substituted by alkyl or acyl.

A bioisostere of an acidic functional group is a functional group which has the same biological properties as an acidic functional group. One example of such a bioisostere is two neighbouring fluoro.

Heteroaryl is a 5- or 6-membered heterocyclic monocyclic group. Such a monocyclic heteroaryl group includes, for example, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-oxadiazol-3-yl, 1,2,4-oxadiazol-5-yl, 1,2,4-thiadiazol-3-yl, 1,2,4-thiadiazol-5-yl, 1,2,5-oxadiazol-3-yl, 1,2,5-oxadiazol-4-yl, 1,2,5-thiadiazol-3-yl, 1,2,5-thiadiazol-4-yl, 1-imidazolyl, 2-imidazolyl, 4-imidazolyl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyrimidinyl, 4-pyrimidinyl, 5-pyrimidinyl, 3-pyridazinyl, 4-pyridazinyl, 2-pyrazinyl, 1-pyrazolyl, 3-pyrazolyl, and 4-pyrazolyl.

A 5-8 membered ring optionally containing double bonds and optionally containing one or two heteroatoms includes for example pyrrolidine, piperidine, piperazine, morpholine, cyclohexyl, cyclohexen, dihydropyrrole, dihydrofuran, dihydrothiophen, dihydropyridine, dihydropyridazine, dihydropyrimidine, dihydropyrazine, tetrahydropyrid ine, tetrahydropyridazine, tetrahydropyrimid ine, tetrahydropyrazine, homopiperazine, homopiperidine, azacyclooctane.

The compounds of this invention may exist in unsolvated as well as in solvated forms with pharmaceutically acceptable solvents such as water, ethanol and the like. In general, the solvated forms are considered equivalent to the unsolvated forms for the purposes of this invention.

Pharmaceutically Acceptable Salts

The chemical compound for use according to the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a chemical compound of the invention includes alkali metal salts, such as the sodium salt of a chemical compound of the invention containing a carboxy group. In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Prodrugs

The substance used according to the invention may be administered as such or in the form of a suitable prodrug thereof. The term “prodrug” denotes a bioreversible derivative of the drug, the bioreversible derivative being therapeutically substantially inactive per se but being able to convert in the body to the active substance by an enzymatic or non-enzymatic process.

Thus, examples of suitable prodrugs of the substances used according to the invention include compounds obtained by suitable bioreversible derivatization of one or more reactive or derivatizable groups of the parent substance to result in a bioreversible derivative. The derivatization may be performed to obtain a higher bioavailability of the active substance, to stabilize an otherwise unstable active substance, to increase the lipophilicity of the substance administered, etc.

Examples of types of substances which may advantageously be administered in the form of prodrugs are carboxylic acids, other acidic groups and amines, which may be rendered more lipophilic by suitable bioreversible derivatization. As examples of suitable groups may be mentioned bioreversible esters or bioreversible amides. Amino acids are typical examples of substances which, in their unmodified form, may have a low absorption upon administration. Suitable prodrug derivatives of amino acids will be one or both of the above-mentioned types of bioreversible derivatives.

Steric Isomers

The chemical compounds of the present invention may exist in (+) and (−) forms as well as in racemic forms. The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or I- (tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

Pharmaceutical Compositions

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers therefor, and, optionally, other therapeutic and/or prophylactic ingredients, know and used in the art. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depend on the nature and severity of the disease being treated and the route of administration, and is within the discretion of the physician, and may be varied by titration of the dosage to the particular circumstances of this invention to produce the desired therapeutic effect. However, it is presently contemplated that pharmaceutical compositions containing of from about 0.01 to about 500 mg of active ingredient per individual dose, preferably of from about 0.1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.01 μg/kg i.v. and 0.1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

Any possible combination of two or more of the embodiments described herein is comprised within the scope of the present invention.

The following example will illustrate the invention further, however, it is not to be construed as limiting.

### EXAMPLES

### Example 1

In vitro Inhibition of Anti-IgE Induced Basophil Histamine Release

The principle: By this method the ability of a test compound to inhibit anti-IgE induced basophil histamine release is measured.

Test compound: The test compound is dissolved in DMSO (stock solution: 100 mM). Three concentration levels of test compound are used (end concentrations: 30, 10, and 3 μM).

Blood samples: Heparinized whole blood is obtained from healthy non allegic blood donors.

Method: Heparinized whole blood is washed and the plasma substituted with a Pipes buffer containing physiologically concentrations of NaCl and KCl.

The washed whole blood is pre-incubated with the compound (above concentrations) in 15 min at 37° C., Anti-IgE is added (three concentration levels of Anti-IgE (100, 30 and 10 U/ml), and the samples are incubated 60 min at 37° C.

The amount of released histamine is measured using the glass-fiber based method (LHRT) (HR-Test Kit from RefLab, Copenhagen, Denmark).

% inhibition is calculated as follows:

$$\frac{histamine release when test compound is present}{histamine release when test compound is not present} \times 100 \%$$

1. The use of a compound of the general formula I
A-(X)ₚ—(Y)_{q}-(Z)ᵣ-B (I)
wherein
A represents a first ring structure selected from aryl or heteroaryl;
which first ring structure is optionally substituted with one or more substituents independently selected from the group consisting of:
halogen, hydroxy, amino, oxy, cyano, nitro, trifluoromethyl, trifluoromethoxy, trifluorothiomethoxy alkyl, cycloalkyl, cycloalkylalkyl, alkenyl, alkynyl, alkoxy, aryl, arylalkyl, aryloxy, arylcarboxy, heteroaryl, —N(R²)-aryl, a 5- or 6-membered monocyclic heterocyclic group, —CO₂R₂, —COR¹, -alkyl-CO₂R¹, -alkyl-COR¹, —N(R²)₂, -alkyl-N(R²)₂, —CO₂N(R¹)₂, —NHCOR¹, —CON(R¹)₂, —NHSO₂R¹, —CONHSO₂R¹, —SO₂N(R¹)₂, and —SO₂OR¹;
wherein each of the alkyl, alkoxy, and cycloalkyl is optionally substituted with one or more substitutents independently selected from the group consisting of:
halogen, hydroxy, amino, cyano, nitro, trifluoromethyl, trifluoromethoxy, trifluorothiomethoxy alkyl, cycloalkyl, cycloalkylalkyl, alkenyl, and alkynyl;
each of the aryl, heteroaryl, and 5- or 6-membered monocyclic heterocyclic group is optionally substituted with one or more one or more substitutents independently selected from the group consisting of:
halogen, hydroxy, amino, cyano, nitro, trifluoromethyl, trifluoromethoxy, trifluorothiomethoxy alkyl, cycloalkyl, cycloalkylalkyl, alkenyl, alkynyl, alkoxy, aryl, heteroaryl, —CO₂R³, —COR³,
—N(R⁴)₂, -alkyl-N(R⁴)₂, —CON(R³)₂, —NHCOR³, —CON(R³)₂, —NHSO₂R³, —SO₂N(R³)₂, and —SO₂OR³;
each of R¹ and R³ independently is selected from the group consisting of:
hydrogen, alkyl, cycloalkyl, cycloalkylalkyl, alkenyl, alkynyl, aryl, arylalkyl, heteroaryl, and
a 5-8 membered ring optionally containing double bonds and optionally containing one or two heteroatoms, which heteroatoms can be substituted with alkyl or acyl;
or (R¹)₂ or (R³)₂ independently together with the heteroatom to which it is connected represents a 5-8 membered ring optionally containing double bonds and optionally containing another heteroatom, which heteroatom can be substituted with alkyl or acyl;
each of R² and R⁴ independently is hydrogen or alkyl;
B represents a second ring structure selected from aryl or heteroaryl;
which second ring structure is substituted with one or more acidic functional group having a pKa value below 8, or a group which is convertible in vivo to such a group, or a bioisostere thereof;
and which second ring structure is furthermore optionally substituted with one or more substituents independently selected from the group consisting of:
halogen, hydroxy, amino, oxy, cyano, nitro, trifluoromethyl, trifluoromethoxy, trifluorothiomethoxy alkyl, cycloalkyl, cycloalkylalkyl, alkenyl, alkynyl, alkoxy, aryl, arylalkyl, aryloxy, arylcarboxy, heteroaryl, —N(R⁶)-aryl, a 5- or 6-membered monocyclic heterocyclic group, —CO₂R⁵, —COR⁵, -alkyl-CO₂R⁵, -alkyl-COR⁵, —N(R⁶)₂, -alkyl-N(R⁵)₂, —CON(R⁵)₂, —NHCOR⁵, —CON(R⁵)₂, —NHSO₂R⁵, —CONHSO₂R⁵, —SO₂N(R⁵)₂, and —SO₂OR⁵;
wherein each of the alkyl, alkoxy, and cycloalkyl is optionally substituted with one or more substitutents independently selected from the group consisting of:
halogen, hydroxy, amino, cyano, nitro, trifluoromethyl, trifluoromethoxy, trifluorothiomethoxy alkyl, cycloalkyl, cycloalkylalkyl, alkenyl, and alkynyl;
each of the aryl, heteroaryl, and 5- or 6-membered monocyclic heterocyclic group is optionally substituted with one or more one or more substitutents independently selected from the group consisting of:
halogen, hydroxy, amino, cyano, nitro, trifluoromethyl, trifluoromethoxy, trifluorothiomethoxy alkyl, cycloalkyl, cycloalkylalkyl, alkenyl, alkynyl, alkoxy, aryl, heteroaryl, —CO₂R⁷, —COR⁷,
—N(R⁸)₂, -alkyl-N(R⁸)₂, —CO₂N(R⁷)₂, —NHCOR⁷, —CON(R⁷)₂, —NHSO₂R⁷, —SO₂N(R⁷)₂, and —SO₂OR⁷;
each of R⁵ and R⁷ independently is selected from the group consisting of:
hydrogen, alkyl, cycloalkyl, cycloalkylalkyl, alkenyl, alkynyl, aryl, arylalkyl, heteroaryl, and
a 5-8 membered ring optionally containing double bonds and optionally containing one or two heteroatoms, which heteroatoms can be substituted by alkyl or acyl;
or (R⁵)₂ or (R⁷)₂ independently together with the heteroatom to which it is connected represents a 5-8 membered ring optionally containing double bonds and optionally containing another heteroatom, which heteroatom can be substituted by alkyl or acyl;
each of R⁶ and R⁸ independently is hydrogen or alkyl;
X, Y, and Z are independently selected from the group consisting of:
—CO—, —CS—, —SO₂—, —C(═NR⁹)—, —NR¹⁰—, —(CH₂)ₛ—, —O—, —CH₂—NH—, —SO₂—NH—, —CH═CH—, —C≡C—, and —N═CH—;
wherein s is 1, 2, or 3;
R⁹ is hydrogen, alkyl, or cyano;
R¹⁰ is hydrogen or alkyl;
p, q, and r independently are 0 or 1;
the sum p+q+r is 1, 2, or 3;
or —(X)ₚ—(Y)_{q}-(Z)ᵣ- represents 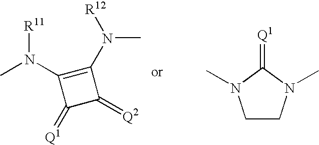
wherein
Q¹ and Q² independently represent O or S;
R¹¹ and R¹² independently are hydrogen or alkyl;
or a pharmaceutically acceptable salt or a prodrug thereof for the manufacture of a medicament for the treatment, prevention or alleviation of a disorder or disease of a subject, which disorder or disease is responsive to modulation of the mast cell or basophil activity of such a subject.

2. The use according to claim 1, wherein the acidic functional group having a pKa below 8, or a group which is convertible in vivo to such a group is selected from the group consisting of:
—COOH, —CH₂CO₂R¹³, —CON(R¹³)₂, tetrazolyl, methyltetrazolyl, 3-oxo-1,2-dihydro-1,2,4-triazolyl, 2-oxo-3H-1,3,4-oxadiazolyl, 3-oxo-1,2-dihydro-1,2,4-triazolyl, 4-hydro-1,2,4-triazolyl, —NHSO₂R¹³, —CO₂R¹³, —CO₂N(R¹³)₂, —SO₂OR¹³, —SO₂N(R³)₂, —CONHOH, —CONHNH₂, —CONHSO₂R¹³, —CONHSO₂OR¹³, —PO(OR¹³)₂, and —SO₂OR¹³;
wherein each of R¹³ independently is selected from the group consisting of:
hydrogen, alkyl, cycloalkyl, cycloalkylalkyl, alkenyl, alkynyl, aryl, aralkyl, and heteroaryl;
or R¹³ comprises a 5-8 membered ring optionally containing double bonds and optionally containing one or two heteroatoms, which heteroatoms can be substituted by alkyl or acyl;
or (R¹³)₂ together with the heteroatom to which it is connected represents a 5-8 membered ring optionally containing double bonds and optionally containing another heteroatom, which heteroatoms can be substituted by alkyl or acyl;
and the bioisostere thereof is two neighbouring fluoro.

3. The use according to claims 1 or 2, wherein the first ring structure is optionally substituted with one or more substituents independently selected from the group consisting of:
trifluoromethyl, halogen, alkyl, alkoxy, nitro, —COR¹, —COOH, —CH₂CO₂R¹, —CON(R¹)₂, —NHSO₂R¹, —NHCOR¹, —CO₂R¹, —CO₂N(R¹)₂, —SO₂N(R¹)₂, —CONHSO₂R¹, —SO₂OR¹, and aryl;
wherein the aryl optionally is substituted with one or more substituents selected from the group:
—NO₂, —NHCOR³, —CO₂R³, —CON(R³)₂, —NHSO₂R³, and —SO₂N(R³)₂;
wherein R¹ and R³ are defined as above.

4. The use according to any one of the claims 1-3 wherein the second ring structure is substituted with one or more acidic functional group having a pKa value below 8, or a group that is convertible in vivo to such a group, or a bioisostere thereof; and which second ring structure is furthermore optionally substituted with one or more substituents independently selected from the group consisting of:
alkyl, nitro, amino, alkylamino, CO₂R⁹, CF₃, alkyl, halogen, hydroxy, alkoxy, —NHCOR⁵, —N(R⁵)₂, —CON(R⁵)₂, and aryl,
wherein the aryl is optionally substituted with one or more substituents independently selected from the group consisting of:
—NO₂, —CON(R⁷)₂, —NHCOR⁷, —SO₂N(R⁷)₂, and —CO₂R⁷;
wherein R⁵ and R⁷ are defined as above.

5. The use according to any one of the claims 1-4, wherein the first ring structure is phenyl;
the second ring structure is phenyl; and
—(X)ₚ—(Y)_{q}-(Z)ᵣ- represents —NH—CO—NH—.

6. The use according to any one of the claims 1-5, wherein the disorder or disease that is responsive to modulation of the mast cell or basophil activity is a disorder or disease that is responsive to modulation of mast cell or basophil production or secretion of histamine.

7. The use according to any one of the claims 1-5, wherein the disorder or disease that is responsive to modulation of the mast cell or basophil activity is allergic bronchopulmonary aspergillosis (ABPA), allergic rhinitis, allergic skin disease, allergic skin reaction, drug induced allergic skin reaction, anaphylaxis, asthma, atherosclerosis, atopic dermatitis (AD), bronchial asthma, cancer, chronic obstructive pulmonary disease (COPD), Chrohn's disease, contact dermatitis, dilated cardiomyopathy, fatal asthma, graft rejection, hypersensitivity pneumonitis, ischemic hearth disease, pulmonary fibrosis, rheumatoid arthritis, systemic sclerosis, urticaria, or uveoretinitis.

8. The use according to claim 7, wherein the disorder or disease that is responsive to modulation of the mast cell or basophil activity is allergic bronchopulmonary aspergillosis (ABPA), allergic rhinitis, allergic skin disease, allergic skin reaction, drug induced allergic skin reaction, asthma, bronchial asthma, fatal asthma and chronic obstructive pulmonary disease (COPD).

9. The use according to any one of the claims 1-8 wherein the compound is selected from
3-Trifluoromethylphenyl-N′-2-carboxyphenyl urea
N-3-Trifluoromethylphenyl-N′-3-carboxyphenyl urea;
N-(2-Methoxy-5-chlorophenyl)-N′-3-carboxyphenyl urea;
N-3-Trifluoromethylphenyl-N′-(2-carboxy-5-nitrophenyl) urea;
N-3-Trifluoromethylphenyl-N′-(2-carboxy-4-methylphenyl) urea;
N-3-Trifluoromethylphenyl-N′-(4-bromo-2-carboxyphenyl) urea;
N-3-Trifluoromethylphenyl-N′-3-carbamoylphenyl urea;
N-3-Trifluoromethylphenyl-N′-3-sulfamoylphenyl urea;
N-3-Trifluoromethylphenyl-N′-(5-chloro-2-phenylsulfonamidocarbonylphenyl) urea;
N-3-Trifluoromethylphenyl-N′-2-methylsulfonamidocarbonylphenyl urea;
N-3-Trifluoromethylphenyl-N′-(6-methyl-2-carboxyphenyl) urea;
N-3-Trifluoromethylphenyl-N′-(3-methyl-2-carboxyphenyl) urea;
N-3-Trifluoromethylphenyl-N′-(4-hydroxy-2-carboxyphenyl) urea;
N-4-Nitrophenyl-N′-2-carboxyphenyl urea;
N-3-Trifluoromethylphenyl-N′-2-carboxymethylphenyl urea;
N-3-Trifluoromethylphenyl-N′-2-sulfophenyl urea;
N-3-Trifluoromethylphenyl-N′-2-carboxyphenyl thiourea;
N-3-Trifluoromethylphenyl-N′-(2-carboxy-5-trifluoromethylphenyl) urea;
N-3-Trifluoromethylphenyl-N′-(4,5-dimethoxy-2-carboxyphenyl) urea;
N-3-Carboxyphenyl-N′-(2-hydroxy-5-chlorophenyl) urea;
N-3-Carbamoylphenyl-N′-(2-hydroxy-5-chlorophenyl) urea;
N-3-Trifluoromethylphenyl-N′-(2-hydroxy-4-nitro-5-carboxyphenyl) urea;
N-3-Trifluoromethylphenyl-N′-(4-carboxy-5-chloro-2-hydroxyphenyl) urea;
N-3-Trifluoromethylphenyl-N′-2-amino-5-chlorophenyl) urea;
N-3-Trifluoromethylphenyl-N′-(5-chloro-2-methanesulfonylaminophenyl) urea;
N-3-Trifluoromethylphenyl-N′-2-carboxyphenyl urea isopropyl ester;
N-3-Trifluoromethylphenyl-N′-2-carboxyphenyl urea methyl ester;
N-3-Trifluoromethylphenyl-N′-2-hydrazinocarbonylphenyl urea;
N-3-Trifluoromethylphenyl-N′-2-hydroxylaminocarbonylphenyl urea;
2-(3′-Trifluoromethylbenzylcarboxamido)benzoic acid;
N-3-Trifluoromethylphenyl-N′-4-carboxyphenyl urea;
N-3-Trifluoromethylphenyl-N′-(2-carboxy-4-nitrophenyl) urea;
N-3-Trifluoromethylphenyl-N′-2-carboxynapht-3-yl urea;
N-3-Trifluoromethylphenyl-N′-(4-methoxy-2-carboxyphenyl) urea;
N-3-Methoxyphenyl-N′-2-carboxyphenyl urea;
N-4-Bromophenyl-N′-2-carboxyphenyl urea;
N-3-Nitrophenyl-N′-2-carboxyphenyl urea;
N-2-Methoxyphenyl-N′-2-carboxyphenyl urea;
N-4-Methoxyphenyl-N′-2-carboxyphenyl urea;
N-1-Naphthyl-N′-2-carboxyphenyl urea;
N-2-Trifluoromethylphenyl-N′-2-carboxyphenyl urea;
N-4-Methylphenylsulfonyl-N′-2-carboxyphenyl urea;
N-3-Trifluoromethylphenyl-N′-(2-ethyloxycarbonylphenyl)-1,2-diaminoethane;
N-(3-Trifluoromethyl)phenyl-N′-(2-carboxy)phenylsulfamide;
N-3-Trifluoromethylbenzyl-N′-2-carboxyphenyl urea;
N-(3-Trifluoromethyl-4-phenylphenyl)-N′-2-carboxyphenyl urea;
2-(3′-Trifluoromethylphenyloxycarbonylamino)benzoic acid;
N-3-Trifluoromethylphenyl-N′-(5-chloro-2-aminophenyl) urea;
N-3-Trifluoromethylphenyl-N′-[4-nitro-2-(1-H-tetrazol-5-yl)phenyl] urea;
N-3-Trifluoromethylphenyl-N′-[4-(2-naphthyl)-2-(1-H-tetrazol-5-yl)phenyl] urea;
N-3-Trifluoromethylphenyl-N′-[4-(3-pyridyl)-2-(1-H-tetrazol-5-yl)phenyl] urea;
N-3-Trifluoromethylphenyl-N′-[4-(1-naphthyl)-2-(1-H-tetrazol-5-yl)phenyl] urea; N-3-Trifluoromethylphenyl-N′-[4-(4-trifluoromethylphenyl)-2-(1-H-tetrazol-5-yl)phenyl] urea;
N-3-Trifluoromethylphenyl-N′-[4-(3-furyl-2-(1-H-tetrazol-5-yl)phenyl] urea;
N-3-Trifluoromethylphenyl-N′-[4-(3-thienyl)2-(1-H-tetrazol-5-yl)phenyl] urea;
N-3-Trifluoromethylphenyl-N′-[4-(3-nitrophenyl)-2-(1-H-tetrazol-5-yl)phenyl] urea;
N-3-Trifluoromethylphenyl-N′-[4-(4-ethoxycarbonylphenyl)-2-(1-H-tetrazol-5-yl)phenyl] urea;
N-3-Trifluoromethylphenyl-N′-[4-(4-dimethylaminocarbonylphenyl)-2-(1-H-tetrazol-5-yl)phenyl] urea;
N-3-Trifluoromethylphenyl-N′-[4-(4-aminocarbonylphenyl)-2-(1-H-tetrazol-5-yl)phenyl] urea;
N-3-Trifluoromethylphenyl-N′-2-(4-hydroxy-1,2,4-triazol-3-yl)phenyl urea;
N-3-Trifluoromethylphenyl-N′-2-(3-oxo-1,2-dihydro-1,2,4-triazol-1-yl)phenyl urea;
N-3-Trifluoromethylphenyl-N′-2-(2-oxo-3H-1,3,4-oxadiazol-5-yl)phenyl urea;
N-3-Trifluoromethylphenyl-N′-[5-phenyl-2-(3-oxo-1,2-dihydro-1,2,4-triazol-1-yl)phenyl] urea;
N-3-Trifluoromethylphenyl-N′-[4-amino-2-(1-H-tetrazol-5-yl)phenyl] urea;
N-3-Trifluoromethylphenyl-N′-[4-acetylamino-2-(1-H-tetrazol-5-yl)phenyl] urea;
N-3-Trifluoromethylphenyl-N′-[4-benzoylamino-2-(1-H-tetrazol-5-yl)phenyl] urea;
N-3-Trifluoromethylphenyl-N′-[4-(4-carboxyphenyl)-2-(1-H-tetrazol-5-yl)phenyl] urea;
N-3-Trifluoromethylphenyl-N′-[4-(4-anilinocarbonylphenyl)-2-(1-H-tetrazol-5-yl)phenyl] urea;
N-4-Biphenylyl-N′-2-1-H-tetrazol-5-yl)phenyl urea;
N-3-Biphenylyl-N′-2-(1-H-tetrazol-5-yl)phenyl urea;
N-5-Indanyl-N′-2-(1-H-tetrazol-5-yl)phenyl urea;
N-3-Bromophenyl-N′-[4-bromo-2-(1-H-tetrazol-5-yl)phenyl] urea;
N-3-Acetylphenyl-N′-2-(1-H-tetrazol-5-yl)phenyl urea;
N-3-Biphenylyl-N′-[4-bromo-2-(1-H-tetrazol-5-yl)phenyl] urea;
N-3-(3-Pyridyl)phenyl-N′-[4-bromo-2-(1-H-tetrazol-5-yl)phenyl] urea;
N-3-Trifluoromethylphenyl-N′-[4-bromo-2-(1-H-tetrazol-5-yl)phenyl urea];
N-3-Trifluoromethylphenyl-N′-2-(1-H-tetrazol-5-yl)phenyl urea;
N-3-Trifluoromethylphenyl-N′-2-(1-H-tetrazol-5-yl)phenyl thiourea;
N-3-Trifluoromethylphenyl-N′-[4-phenyl-2-(1-H-tetrazol-5-yl)phenyl] urea;
N-4-Trifluoromethylphenyl-N′-2-(1-H-tetrazol-5-yl)phenyl urea;
N-3-Chlorophenyl-N′-2-(1-H-tetrazol-5-yl)phenyl urea;
N-Phenyl-N′-2-(1-H-tetrazol-5-yl)phenyl urea;
N-3-Bromophenyl-N′-[4-bromo-2-(1-H-tetrazol-5-yl)phenyl] urea;
3-[4-Bromo-2-(1-H-tetrazol-5-yl)-phenylamino]-4-(3-trifluoromethyl-phenylamino)-3-cyclobuten-1,2-dione;
3-(3-Bromo-phenylamino)-[4-bromo-(1-H-tetrazol-5-yl)-phenylamino]-3-cyclobuten-1,2-dione;
3-(3-Bromo-phenylamino)-4-[4′-(N,N-dimethyl sulfonamide)-2-(1-H-tetrazol-5-yl)-biphenylamino]-3-cyclobuten-1,2-dione;
3-(3-Bromo-phenylamino)-4-[2-(1-H-tetrazol-5-yl)-biphenylamino]-3-cyclobuten-1,2-dione;
N-Phenyl-N′-(2-carboxyphenyl) urea;
N-3-Trifluoromethylphenyl-N′-(2-carboxyphenyl)-N′-methyl urea;
N-3-Trifluoromethylphenyl-N′-(4-bromo-2-carboxyphenyl) urea;
N-3-Trifluoromethylphenyl-N′-(2-carboxy-4-chlorophenyl) urea;
N-3-Trifluoromethylphenyl-N′-(2-carboxy-4-fluorophenyl) urea;
N-3-Trifluoromethylphenyl-N′-(5-bromo-2-carboxyphenyl) urea;
N-3-Trifluoromethylphenyl-N′-(2-carboxy-5-chlorophenyl) urea;
N-3-Bromophenyl-N′-[2-(1-H-tetrazol-5-yl)-4-biphenyl] urea;
N-3-Trifluoromethylphenyl-N′-[4′-(N,N-dimethylsulfamoyl)-2-(1-H-tetrazol-5-yl)-4-biphenyl] urea;
N-3-Bromophenyl-N′-[4′-(N,N-dimethylsulfamoyl)-2-(1-H-tetrazol-5-yl)-4-biphenyl] urea;
N-3-Bromophenyl-N′-[4′-(N,N-dimethylcarbamoyl)-2-(1-H-tetrazol-5-yl)-4-biphenyl] urea;
N-3-Trifluoromethylphenyl-N′-[4-amino-2-(1-H-tetrazol-5-yl)phenyl] urea;
N-3-Trifluoromethylphenyl-N′-[4-acetylamino-2-(1-H-tetrazol-5-yl)phenyl] urea;
N-3-Trifluoromethylphenyl-N′-[4′-carbamoyl-2-(1-H-tetrazol-5-yl)-4-biphenyl] urea;
N-3-Trifluoromethylphenyl-N-[4′-(N,N-dimethylcarbamoyl)-2-(1-H-tetrazol-5-yl)-4-biphenyl] urea;
N-3-Trifluoromethylphenyl-N′-[4′-carboxy-2-(1-H-tetrazol-5-yl)-4-biphenyl] urea;
1-(3-Trifluoromethylphenyl)-3-(2-carboxyphenyl)-2-imidazolidone;
N-3-Trifluoromethylphenyl-N′-[4-(4-benzoylcarbonylphenyl)-2-(1-H-tetrazol-5-yl)phenyl] urea;
N-4-Biphenylyl-N′-2-(1-H-tetrazol-5-yl)phenyl urea;
N-3-Bromophenyl-N′-[3′-nitro-2-(1-H-tetrazol-5-yl)biphenyl] urea;
N-3-Bromophenyl-N′-[4′-(sulfoamido-N-methylpiperazinium chloride)-2-(1-H-tetrazol-5-yl)-4-biphenyl] urea;
N-3-Bromophenyl-N-[4′-(carbamoyl-N-methylpiperazine)-2-(1-H-tetrazol-5-yl)-4-biphenyl] urea;
N-3,5-Dichlorophenyl-N′-[4-bromo-2-(1-H-tetrazol-5-yl)phenyl] urea;
N-4-Trifluoromethylphenyl-N′-[4-bromo-2-(1-H-tetrazol-5-yl)phenyl] urea;
N-4-Bromophenyl-N′-[4-bromo-2-(1-H-tetrazol-5-yl)phenyl] urea;
N-3-Methoxyphenyl-N′-[4-bromo-2-(1-H-tetrazol-5-yl)phenyl] urea;
N-3-Chlorophenyl)-N′-[4-bromo-2-(1-H-tetrazol-5-yl)phenyl] urea;
N-3-Methylphenyl-N-[4-bromo-2-(1-H-tetrazol-5-yl)phenyl] urea;
N-3,4-Dichlorophenyl-N′-[4-bromo-2-(1-H-tetrazol-5-yl)phenyl] urea;
N-2-Naphthyl-N-[4-bromo-2-(1-H-tetrazol-5-yl)phenyl] urea;
N-(4-Methyl-3-nitrophenyl)-N′-[4-bromo-2-(1-H-tetrazol-5-yl)phenyl] urea;
N-(2-Chloro-4-trifluoromethylphenyl)-N′-[4-bromo-2-(1-H-tetrazol-5-yl)phenyl] urea;
N-3,5-Di(trifluoromethyl)phenyl-N′-[4-bromo-2-(1-H-tetrazol-5-yl)phenyl] urea;
N-3,5-Dimethylphenyl-N-[4-bromo-2-(1-H-tetrazol-5-yl)phenyl] urea;
N-4-Ethoxyphenyl-N′-[4-bromo-2-(1-H-tetrazol-5-yl)phenyl] urea;
N-4-Methoxyphenyl-N′-[4-bromo-2-(1-H-tetrazol-5-yl)phenyl] urea;
N-2-Trifluoromethylphenyl-N′-[4-bromo-2-(I-H-tetrazol-5-yl)phenyl] urea;
N-2-Bromophenyl-N′-[4-bromo-2-(1-H-tetrazol-5-yl)phenyl] urea;
N-2-Chlorophenyl-N′-[4-bromo-2-(1-H-tetrazol-5-yl)phenyl] urea;
N-2-Fluorophenyl-N′-[4-bromo-2-(1-H-tetrazol-5-yl)phenyl] urea;
N-(4-Chloro-3-trifluoromethylphenyl)-N′-[4-bromo-2-(1-H-tetrazol-5-yl)phenyl] urea;
N-3-Bromophenyl-N′-2,3-difluorophenyl urea;
N-2-Methylphenyl-N-[4-bromo-2-(1-H-tetrazol-5-yl)phenyl] urea;
N-2-Ethylphenyl-N′-[4-bromo-2-(1-H-tetrazol-5-yl)phenyl] urea;
N-4-Methylphenyl-N-[4-bromo-2-(1-H-tetrazol-5-yl)phenyl] urea;
N-2-Nitrophenyl-N′-[4-bromo-2-(1-H-tetrazol-5-yl)phenyl] urea;
N-3-Fluorophenyl-N′-[4-bromo-2-(1-H-tetrazol-yl)phenyl] urea;
N-4-(2-Propyl)phenyl-N-[4-bromo-2-(1-H-tetrazol-5yl)phenyl] urea;
N-3-Nitrophenyl-N-[4-bromo-2-(1-H-tetrazol-5-yl)phenyl] urea;
N-3-Acetylphenyl-N′-[4-bromo-2-(1-H-tetrazol-5-yl)phenyl] urea;
N-4-Nitrophenyl-N′-[4-bromo-2-(1-H-tetrazol-5-yl)phenyl] urea;
N-3-Trifluoromethylphenyl-N′-2-carboxyphenyl urea;
N-Phenyl-N-2-carboxyphenyl urea;
N-3-Trifluoromethylphenyl-N′-2-carboxyphenyl-N-methyl urea;
N-3-Trifluoromethylphenyl-N′-[4′-(N-phenylcarbamoyl)-2-(1-H-tetrazol-5-yl)-4-biphenyl] urea;
N-(2-Indan)-N′-2-(1-H-tetrazol-5-yl)phenyl urea;
N-(4-Biphenyl)-N′-2-(1-H-tetrazol-5-yl)phenyl urea;
N-(3-Biphenyl)-N′-2-(1-H-tetrazol-5-yl)phenyl urea;
N-(3-Acetylphenyl)-N′-2-(1-H-tetrazol-5-yl)phenyl urea;
N-3-Trifluoromethylphenyl-N′-[2-(1-methyltetrazol-5-yl)-4-biphenyl] urea;
N-(3-Biphenyl)-N′-[4-bromo-2-(1-H-tetrazol-5-yl)phenyl] urea;
N-3-(3-Pyridyl)phenyl-N′-[4bromo-2-(1-H-tetrazol-5-yl)phenyl] urea hydrochloride;
N-3-Bromophenyl-N′-[4-bromo-2-(1-H-tetrazol-5-yl)phenyl] urea;
1-3-Trifluoromethylphenyl)-3-(2-carboxyphenyl)-2-imidazolidone;
N-(4-Biphenylyl)-N′-2-(1-H-tetrazol-5-yl)phenyl urea;
N-(3-Biphenylyl)-N′-2-(1-H-tetrazol-5-yl)phenyl urea;
N-(5-Indanyl)-N′-2-(1-H-tetrazol-5-yl)phenyl urea;
N-(2-Chloro-5-trifluoromethylphenyl)-N′-[4-bromo-2-(1-H-tetrazol-5-yl)phenyl] urea;
N-4-Bromophenyl-N′-(2-carboxy-5-chlorophenyl) urea;
N-4-Trifluoromethylphenyl-N′-(2-carboxy-5-chlorophenyl) urea;
N-3-Bromophenyl-N′-(2-carboxy-5-chlorophenyl) urea;
N-3-Nitrophenyl-N′-(2-carboxy-5-chlorophenyl) urea;
N-3-Methoxyphenyl-N′-(2-carboxy-5-chlorophenyl) urea;
N-(4-Chloro-3-trifluoromethylphenyl)-N′-(2-carboxy-5-chlorophenyl) urea;
N-3-Fluorophenyl-N′-(2-carboxy-5-chlorophenyl) urea;
N-3-Fluorophenyl-N′-(2-carboxy-5-fluorophenyl) urea;
N-3-Trifluoromethylphenyl-N′-(2-carboxy-4,5-difluorophenyl) urea;
N-3,5-Bis(trifluoromethyl)phenyl)-N′-(2-carboxy-5-chlorophenyl) urea;
N-3-Trifluoromethylphenyl-N′-(2-carboxy-5-nitrophenyl) urea;
N-3,4-Dichlorophenyl-N′-[5-methyl-2-(1-H-tetrazol-5-yl)phenyl] urea;
N-3,4-Dichlorophenyl-N′-[5chloro-2-(1-H-tetrazol-5-yl)phenyl] urea;
N-3-Trifluoromethylphenyl-N′-(3-carboxy-4-chlorophenyl) urea;
N-(3-Chloro-4-hydroxyphenyl)-N′-[4-bromo-2-(1-H-tetrazol-5-yl)phenyl] urea;
N-2,3,4-Trifluorophenyl-N′-[4-bromo-2-(1-H-tetrazol-5-yl)phenyl] urea;
N-3,4-Difluorophenyl-N′-[4-bromo-2-(1-H-tetrazol-5yl)phenyl] urea;
N-3-Chlorophenyl-N′-2,3-difluorophenyl urea;
N-(3-Chloro-4-fluorophenyl)-N′-[4-bromo-2-(1-H-tetrazol-5-yl)phenyl] urea;
N-2,4,5-Trifluorophenyl-N′-[4-bromo-2-(1-H-tetrazol-5-yl)phenyl] urea;
N-3,5-Bis(trifluoromethyl)phenyl)-N′-2-(1-H-tetrazol-5-yl)phenyl urea;
N-3,5-Bis(trifluoromethyl)phenyl)-N′-[2,4-dibromo-6-(1-H-tetrazol-5-yl)phenyl] urea;
N-(4-Fluoro-3-trifluoromethylphenyl)-N′-[4-bromo-2-(1-H-tetrazol-5-yl)phenyl] urea;
N-3,5-Difluorophenyl-N′-[4-bromo-2-(1-H-tetrazol-5-yl)phenyl] urea;
N-3,5-Bis(trifluoromethyl)phenyl-N′-[4′-(N,N-dimethylsulfamoyl)-2-(1-H-tetrazol-5-yl)-(4-biphenyl)] urea;
N-3,5-Dichlorophenyl-N′-[4′-(N,N-dimethylsulfamoyl)-2-(I -H-tetrazol-5-yl)-4-biphenyl] urea;
N-2,3-Difluorophenyl-N′-3-trifluoromethylphenyl urea;
N-2,3-Difluorophenyl-N′-(4-chloro-3-trifluoromethylphenyl) urea;
N-3,4-Dichlorophenyl-N′-2,3-trifluorophenyl urea;
N-2,3-Difluorophenyl-N′-3-trifluoromethylphenyl thiourea;
N-2,3-Difluorophenyl-N′-2-fluorophenyl urea;
N-2,3-Difluorophenyl-N′-3-methoxyphenyl urea;
N-3,4-Dichlorophenyl-N′-2,3,4-trifluorophenyl urea;
N-(4-Chloro-3-trifluoromethylphenyl)-N′-2,3,4-trifluorophenyl urea;
N-3-Chlorophenyl-N′-(2-hydroxy-4-methylphenyl) urea;
N-2,3-Difluorophenyl-N′-[3′-(pyridin-3-yl)phenyl] urea;
N-3,5-Dichlorophenyl-N′-2,3-difluorophenyl urea;
N-2,3-Difluorophenyl-N′-3-nitrophenyl urea; and
pharmaceutically acceptable salts and prodrugs thereof.

10. A method of treatment, prevention or alleviation of a disorder or disease of a subject, which disorder or disease is responsive to modulation of the mast cell or basophil activity of such a subject, which method comprises administering to said subject a therapeutically effective amount of a compound of general formula I as defined in claim 1 or a pharmaceutically acceptable salt or a prodrug thereof.
